SEQUENCE LISTING (5 sequences)

SEQ ID NO: 1 - DNA, 1932 bp, Homo sapiens
atggggctcctgcccaagctcggcgtgtcccagggcagcgacacctctactagccgagccggccgctgtgcccgctcggtcttcggcaacattaaggtgtttgtgctctgccaaggcctcctgcagctctgccaactcctgtacagcgcctacttcaagagcagcctcaccaccattgagaagcgctttgggctctccagttcttcatcgggtctcatttccagcttgaatgagatcagcaatgccatcctcatcatctttgtcagctactttggcagccgggtgcaccgtccacgtctgattggcatcggaggtctcttcctggctgcaggtgccttcatcctcaccctcccacacttcctctccgagccctaccagtacaccttggccagcactgggaacaacagccgcttgcaggccgagctctgccagaagcattggcaggacctgcctcccagtaagtgccacagcaccacccagaacccccagaaggagaccagcagcatgtggggcctgatggtggttgcccagctgctggctggcatcgggacagtgcctattcagccatttgggatctcctatgtggatgacttctcagagcccagcaactcgcccctgtacatctccatcttatttgccatctctgtatttggaccggctttcgggtacctgctgggctctatcatgctgcagatctttgtggactatggcagggtcaacacagctgcagttaacttggtcccgggtgacccccgatggattggagcctggtggctaggcctgctcatttcttcagctttattggttctcacctctttccccttttttttcttccctcgagcaatgcccataggagcaaagagggctcctgccacagcagatgaagcaaggaagttggaggaggccaagtcaagaggctccctggtggatttcattaaacggtttccatgcatctttctgaggctcctgatgaactcactcttcgtcctggtggtcctggcccagtgcaccttctcctccgtcattgctggcctctccaccttcctcaacaagttcctggagaagcagtatggcacctcagcagcctatgccaacttcctcattggtgctgtgaacctccctgctgcagccttggggatgctgtttggaggaatcctcatgaagcgctttgttttctctctacaaaccattccccgcatagctaccaccatcatcaccatctccatgatcctttgtgttcctttgttcttcatgggatgctccaccccaactgtggccgaagtctacccccctagcacatcaagttctatacatccgcagtctcctgcctgccgcagggactgctcgtgcccagattctatcttccacccggtctgtggagacaatggaatcgagtacctctccccttgccatgccggctgcagcaacatcaacatgagctctgcaacctccaagcaactgatctatttgaactgcagctgtgtgaccgggggatccgcttcagcaaagacaggatcgtgccctgtcccctgtgcccacttcctgctcccggccatcttcctcatctccttcgtgtccctgatagcctgcatctcccacaaccccctctacatgatggttctgcgtgtggtgaaccaggaggaaaagtcatttgccatcggggtgcagttcttgttgatgcgcttgctggcctggctgccatctccagccctctatggcctcaccattgaccactcctgcatccggtggaactcgctgtgcttggggaggcgaggggcctgcgcctactatgacaacgatgctctccgagacaggtacctgggcctgcagatgggctacaaggcgctgggcatgctgctgctttgcttcatcagctggagggtgaagaagaacaaggagtacaacgtgcagaaggcggcaggcctcatctga

SEQ ID NO: 2 - DNA, 1038 bp, Homo sapiens
atggggctcctgcccaagctcggcgtgtcccagggcagcgacacctctactagccgagccggccgctgtgcccgctcggtcttcggcaacattaaggtgtttgtgctctgccaaggcctcctgcagctctgccaactcctgtacagcgcctacttcaagagcagcctcaccaccattgagaagcgctttgggctctccagttcttcatcgggtctcatttccagcttgaatgagatcagcaatgccatcctcatcatctttgtcagctactttggcagccgggtgcaccgtccacgtctgattggcatcggaggtctcttcctggctgcaggtgccttcatcctcaccctcccacacttcctctccgagccctaccagtacaccttggccagcactggcagcacatcaagttctatacatccgcagtctcctgcctgccgcagggactgctcgtgcccagattctatcttccacccggtctgtggagacaatggaatcgagtacctctccccttgccatgccggctgcagcaacatcaacatgagctctgcaacctccaagcaactgatctatttgaactgcagctgtgtgaccgggggatccgcttcagcaaaggcaggatcgtgccctgtcccctgtgcccacttcctgctcccggccatcttcctcatctccttcgtgtccctgatagcctgcatctcccacaaccccctctacatgatggttctgcgtgtggtgaaccaggaggaaaagtcatttgccatcggggtgcagttcttgttgatgcgcttgctggcctggctgccatctccagccctctatggcctcaccattgaccactcctgcatccggtggaactcgctgtgcttggggaggcgaggggcctgcgcctactatgacaacgatgctctccgagacaggtacctgggcctgcagatgggctacaaggcgctgggcatgctgctgctttgcttcatcagctggagggtgaagaagaacaaggagtacaacgtgcagaaggcggcaggcctcatctga

SEQ ID NO: 3 - protein, 345 aa, Homo sapiens
MGLLPKLGVSQGSDTSTSRAGRCARSVFGNIKVFVLCQGLLQLCQLLYSAYFKSSLTTIEKRFGLSSSSSGLISSLNEISNAILIIFVSYFGSRVHRPRLIGIGGLFLAAGAFILTLPHFLSEPYQYTLASTGSTSSSIHPQSPACRRDCSCPDSIFHPVCGDNGIEYLSPCHAGCSNINMSSATSKQLIYLNCSCVTGGSASAKAGSCPVPCAHFLLPAIFLISFVSLIACISHNPLYMMVLRVVNQEEKSFAIGVQFLLMRLLAWLPSPALYGLTIDHSCIRWNSLCLGRRGACAYYDNDALRDRYLGLQMGYKALGMLLLCFISWRVKKNKEYNVQKAAGLI

SEQ ID NO: 4 - DNA, 30 bp, Artificial Sequence
  Primer of exon 1 derived from human lung PGT for PCR test
tcctgcccaagctcggcgtgtcccagggca

SEQ ID NO: 5 - DNA, 30 bp, Artificial Sequence
  Primer of exon 14 derived from human lung PGT for PCR test
accctccagctgatgaagcaaagcagcagc

(19) United States
(12) Patent Application Publication
(10) Pub. No.: US 2003/0032793 A1
(43) Pub. Date: Feb. 13, 2003

(54) Gene of PGT of human brain type

(76) Inventors: Toru Kawamura, Tokyo (JP); Satoshi Horie, Tokyo (JP); Toshiaki Akira, Tokyo (JP); Shusei Uno, Tokyo (JP)

Correspondence Address: SUGHRUE MION, PLLC, 2100 PENNSYLVANIA AVENUE, N.W., WASHINGTON, DC, 20037, US

(21) Appl. No.: 10/115,126

(22) Filed: Apr. 4, 2002

Related U.S. Application Data
(63) Continuation-in-part of application No. 10/048,289.
Application No. 10/048,289 is a 371 of international application No. PCT/JP00/05103, filed on Jul. 28, 2000.

(30) Foreign Application Priority Data
Jul. 30, 1999 (JP) 11-217845

(51) Int. Cl. C07K 14/435; C07H 21/04

(52) U.S. Cl. 536/23.5; 530/350

(57) ABSTRACT

It is the matter to be solved by the present invention to provide mRNA coding for the PGT of a human brain type and to provide a polynucleotide coding for the PGT of a human brain type prepared using the said mRNA as a template. There are provided mRNA coding for the PGT of a human brain type and having a molecular weight of 38-40 kilodaltons and a polynucleotide being prepared by the use of the said mRNA as a template and having a specific base sequence represented by SEQ ID NO: 2 of the Sequence Listing.

TECHNICAL FIELD

The present invention relates to a polynucleotide being concerned with a PGT (prostaglandin transporter) of a human brain type and also to a polypeptide corresponding to the sequence thereof. More particularly, it relates to mRNA coding for the PGT of a human brain type, to a polynucleotide prepared by using the said mRNA as a template and to a polypeptide corresponding to the sequence thereof.

### BACKGROUND OF THE INVENTION

Prostaglandin transporter (PGT) has a property of transportation of prostaglandin compounds in vivo in a carrier-mediated manner. Prostaglandin permeates through a cellular membrane mediated by PGT to express a physiological activity in the cells thereof or to be metabolized/degraded by enzyme and the like. PGT has been known to be present in lung, kidney, brain, etc. in human body and, in addition, sequence of the gene coding for PGT has been confirmed as well (Science, Volume 268, pages 866-868, 1995). However, the PGT where its genetic sequence has been confirmed is that derived from lung while there has been no report for detailed functions and for a gene concerning a PGT of a brain type.

Further, according to the recent report, a relation of PGT with apoptosis (cell death) or particularly with apoptosis in neurons was pointed out (J. Neurochemistry, Volume 72, No. 5, pages 1907-1914, 1999) and participation of the PGT of a human brain type in diseases caused by apoptosis of neurons has been receiving public attention.

As a result of the rapid progress of genetic engineering in recent years, it is now possible that DNA coding for a desired protein is induced into vector by means of a recombination technique for DNA and then it is integrated with various microbes (such as Escherichia coli, yeast, Bacillus subtilis, etc.), animal cells and the like whereby the said protein (physiologically active substance, various hormones, interferon, cytokine, vaccine, plasma protein, plasminogen activating factor, etc.) is produced in large quantities, in an efficient manner and in an industrial scale.

Clarification of the PGT of a brain type in a level of gene (RNA/DNA) using the above-mentioned means is the first step for the application of genetic engineering and recombination technique to the PGT of a brain type.

### DISCLOSURE OF THE INVENTION

The matter to be solved by the present invention is to provide mRNA coding for the PGT of a human brain type and to provide DNA of a human brain type prepared by using the said mRNA as a template. More particularly, it is to provide mRNA coding for the PGT of a human type, to provide a polynucleotide of the PGT of a human brain type prepared by using the said mRNA as a template and to provide a polypeptide for which the said polynucleotide codes.

As a result of the progress of a study taking the above-mentioned circumstances into consideration, the present inventors have succeeded in collecting the mRNA coding for the PGT of a human brain type. They have further prepared the DNA of the PGT of a human brain type using the said mRNA as a template, analyzed its base sequence and found that it has an amino acid sequence consisting of a new sequence whereupon the present invention has been achieved.

The present invention relates to mRNA coding for the PGT of a human brain type, to a polynucleotide of the PGT of a human brain type prepared by using the mRNA as a template and to the corresponding polypeptide.

### BEST MODE FOR CARRYING OUT THE INVENTION

### DESCRIPTION OF THE DRAWINGS

FIGS. 1 and 2 plot the data of the Experimental example found in this application.

### COLLECTION OF MRNA

The mRNA of the present invention can be collected, for example, by such a manner that human brain cells are cultured, RNA is recovered from the cells having a high PGT-expressing activity, RNA having a poly A sequence is recovered from the said RNA and then the mRNA having the said poly A sequence is collected by a density-gradient centrifugation or the like.

It is also possible that the commercially available human brain poly A (+) RNA is purchased and the mRNA is collected therefrom. It is further possible that the desired mRNA is collected (hooked up) by using the already known gene of the PGT of a human lung type as a primer.

Size of the coding region of the said mRNA is about 1 kb.

(Preparation of DNA)

The said DNA can be prepared by using the above-mentioned mRNA which codes for the PGT of a human brain type as a template. For example, an RNA-DNA hybrid is prepared from the mRNA coding for a human brain type using a reverse transcriptase and then mRNA is removed therefrom whereupon cDNA (complimentary DNA) is prepared. It is possible to prepare ds cDNA from that cDNA using polymerase. As a simple means, an RT-PCR (reverse transcription-polymerase chain reaction) or the like may be used.

The obtained product is transduced into a vector and, using the same, an appropriate host (such as Escherichia coli) is transformed. The transformed host (transformant) is cultured where positive colonies are selected. Then a plasmid DNA is recovered from the selected positive colonies whereupon the base sequence can be analyzed.

As to the base sequence, that which is represented by SEQ ID NO:2 of the Sequence Listing may be exemplified.

The base sequence represented by SEQ ID NO: 2 of the Sequence Listing is a polynucleotide consisting of a base sequence of 1038 bases from 1(A) to 1038(A) having a homology to exons 1-3, GACG and exons 10-14 in the DNA coding for the PGT of a human lung type (SEQ ID NO: 1 of the Sequence Listing).

Incidentally, the relation between each exon of the PGT of a human lung type and the base sequence in SEQ ID NO: 1 of the Sequence Listing is shown as in Table 1.

TABLE 1

| Exon | Base Sequence (SEQ ID NO: 1) |
|---|---|
| 1 | 1 (A)˜96 (G) |
| 2 | 97 (A)˜234 (G) |
| 3 | 235 (A)˜397 (G) |
| 4 | 398 (A)˜625 (T) |
| 5 | 626 (C)˜724 (G) |
| 6 | 725 (C)˜861 (G) |
| 7 | 862 (A)˜940 (C) |
| 8 | 941 (G)˜1105 (G) |
| 9 | 1106 (G)˜1295 (G) |
| 10 | 1296 (C)˜1461 (G) |
| 11 | 1462 (A)˜1626 (T) |
| 12 | 1627 (G)˜1695 (G) |
| 13 | 1696 (C)˜1814 (G) |
| 14 | 1815 (G)˜1932 (A) |

(Polypeptide)

The polypeptide corresponding to the polynucleotide resented by SEQ ID NO: 2 of the Sequence Listing is resented by SEQ ID NO: 3 of the Sequence Listing. Difference of the said amino acid sequence from the PGT of a lung type is that a sequence of 298 amino acids from the 134th Asn to the 431st Pro in the PGT of a lung type is deficient. The amino acid sequence of the present invention is particularly characterized by the sequence of the 133rd Gly and the 134th Ser of SEQ ID NO: 3 of the Sequence Listing. Biological activity of the said PGT of the brain type can be detected by a method (a system where intracellular up-take of PGE1 is measured using HeLa cells) described in Science, Volume 268, pages 866-868, 1995.

(Polynucleotide)

Besides the substance represented by SEQ ID NO: 2, the polynucleotide of the present invention further covers a complementary strand of that sequence and a polynucleotide which hybridizes to the polynucleotide represented by SEQ ID NO: 2 of the Sequence Listing under a stringent condition. Such a polynucleotide is useful as a probe or a primer for the detection of mRNA or gene coding for the PGT, as an antisense oligonucleotide for the regulation of expression of gene, etc.

(Transformant)

The polypeptide of the present invention is provided by means of a genetic recombination technique by utilizing a cell-free protein synthesis system or a host known per se such as Escherichia coli, yeast, Bacillus subtilis, insect cells, animal cells, etc. Known methods per se are applied to the transformation and, for example, a transformation of host is carried out using plasmid, chromosome, virus, etc. as a replicon. Vector is selected depending upon the type of the selected host and its constituting elements are polynucleotide coding for a polypeptide with an object of expression and genetic sequence carrying the information concerning replication and control. Combination is different between prokaryotes and eukaryotes where promoter, binding site of ribosome, terminator, signal sequence, enhancer, etc. may be utilized and combined by a known method per se. Culture of the transformant is carried out by selecting the optimum condition for each host and, with regard to a marker, produced aimed peptide and/or transformant amount may be used.

Recovery of the polypeptide of the present invention from the culture medium may be carried out by a combination of molecular sieve chromatography (gel filtration), ion exchange chromatography, affinity chromatography, etc. using a PGT activity as a marker or by a fractionating means depending upon the difference in solubility. Preferably, there is used a method where, depending upon the information of amino acid sequence, an antibody against the said sequence is prepared and a recovery by a specific adsorption by poly- or monoclonal antibody is carried out.

An antibody is prepared by selecting the antigenic determinant of the polypeptide of the present invention. The antigen is composed of at least 8 or more, preferably 10 or more or, more preferably, 12 or more amino acids. It is preferred that the antibody which is specific to the PGT of a brain type is characterized by the sequence of Gly and Ser which are 133rd and 134th in SEQ ID NO: 3 of the Sequence Listing. The selected antigen is carried out by conducting an immunological induction such as humoral response and/or cellular response to the animal either solely or bonding to a carrier in the presence or absence of adjuvant. There is no particular limitation for the carrier so far as the carrier per se does not cause a harmful action to the host and there may be exemplified cellulose, polymerized amino acid, albumin, etc. With regard to the animal to be immunized, there may be appropriately used mouse, rat, rabbit, goat, horse, etc. Polyclonal antibody is prepared by a method for the recovery of an antibody which is known per se while monoclonal antibody is prepared by such a method where antibody-producing cells are recovered from the animal which was subjected to an immunizing means and then a transformation means which is known per se to immortalized cells is introduced.

The polypeptide, polynucleotide and antibody prepared as such are useful as a marker for a diagnostic means in which the PGT of a human brain type is participated. When at least one of them is utilized, it is possible to construct a screening system for various medicaments. For example, a substance which inhibits the PGT is able to induce apoptosis of neurons. A substance which activates the PGT is able to suppress apoptosis of neurons. It is also possible by means of a screening to prepare a substance which has an affinity to the PGT and is incorporated into cells mediated by the PGT to suppress apoptosis. Accordingly, the polypeptide, polynucleotide and antibody of the present invention and a compound which is prepared by means of a screening utilizing them are useful for the diseases caused by apoptosis of neurons in which PGT is participated such as dementia.

### EXAMPLES

In order to illustrate the present invention in more detail, an example is given as hereunder although the present invention is never limited by that.

### EXAMPLE

About 1 kb of the product was recovered from a human brain poly A(+)RNA (manufactured by Clonetech) by an RT-PCR (reverse transcription polymerase chain reaction) using exon 1 and exon 14 primers of the PGT of a human lung type (DDBJ, U70867) of the already-issued paper (Science, Volume 68, pages 866-868, 1995) concerning cloning of the PGT gene of a human brain type. The recovered PCR product was integrated into a T-vector (PCR 2.1 vector; manufactured by Invitrogen), induced into Escherichia coli (JM-109) and cultured. After that, a plasmid DNA was recovered from positive colonies and a base sequence was determined using a sequencer.

Reaction conditions for the RT-PCR were as follows.

With regard to the RT, primer and oligo dT were made to react under the following condition. Thus, 1) at 30□C for 10 minutes; 2) at 42□C. for 30 minutes; 3) at 99□C. for 5 minutes and then 4) at 5□C. for 5 minutes.

With regard to the PCR, primer of each of exon 1 and exon 14 of the PGT of a human lung type (DDBJ, U70867) was used and the reaction was carried out under the following condition.

Exon 1-primer:

| | |
|---|---|
| Exon 1-primer: | |
| 5′-TCCTGCCCAAGCTCGGCGTGTCCCAGGGCA-3′ | (SEQ ID NO:4 of the Sequence Listing) |
| Exon 14-primer: | |
| 5′-ACCCTCCAGCTGATGAAGCAAAGCAGCAGC-3′ | (SEQ ID NO:5 of the Sequence Listing) |

Thus, 1) at 94□C for 2 minutes; 2) 40 cycles where each comprising at 94□C. for 30 seconds, at 56□C for 30 seconds and 72□C. for 1.5 minutes; and then 3) at 72□C. for 10 minutes.

Incidentally, for the RT-PCR, there was used an RNA PCR kit (manufactured by Takara) and, for the duplication reaction, there was used a DNA thermal cycler (PJ 2000; manufactured by Perkin Elmer).

The DNA coding for the analyzed PGT of a human brain type was found to be a polynucleotide consisting of a base sequence as represented by SEQ ID NO: 2 of the Sequence Listing. This polynucleotide was able to encode the polypeptide consisting of 345 amino acid residues as represented by SEQ ID NO: 3 of the Sequence Listing.

Molecular weight as a protein of the PGT of a human brain type estimated from the amino acid sequence was roughly calculated whereupon it was 38-40 kilodaltons in the amino acid sequence of SEQ ID NO: 3 of the Sequence Listing.

### EXPERIMENTAL EXAMPLE

1.Test Method

1) Construction of PGT 40 and PGT 70 Expression Vectors

PGT 40 or PGT 70 cDNA was inserted into pTracer-SV 40 vector (Invitrogen) to construct an expression vector. PGT 40 means the brain-type PGT of the present invention. PGT 70 means the lung-type PGT which was disclosed in the prior art literature.

2) Rat Cerebral Cortex Neuronal Culture

Fetus of 17 days age (viviparous) was ejected from pregnant rat of SD strain and, in an ice-cold L-15 medium (Invitrogen), cerebral cortex was collected from brain of the fetus. After it was finely cut by scissors, cells were dispersed using a kit for dispersing the neurons (Sumilon), sowed on a polyethyleneimine-coated cover glass (diameter: 12 mm; manufactured by Asahi Technoglass) in a living cell density of 1.6×10⁵ cells/cm² and cultured (at 37° C. with 5% of CO₂). Incidentally, the culture medium used was a Neurobasal medium (Invitrogen) containing 2% of B27 supplement (Invitrogen), 25 μmol/L of glutamic acid (Invitrogen), 0.5 mmol/L of L-glutamine (Invitrogen), 27.5 μg/L of 2-mercaptoethanol (Invitrogen), 100 units/mL of penicillin G (Invitrogen) and 100 μg/L of streptomycin sulfate (Invitrogen).

3) Transfection of PGT 40 and PGT 70 Genes

After each expression vector prepared in 1) was cultured at room temperature for 20 minutes with Lipofectamine 2000 (Invitrogen), it was added to a culture medium of neurons of the third day of the cultivation and then cultured.

4) Apoptosis induction by Amyloid β peptide (Aβ) and inhibiting action to PGE 1

After 24 hours from the initiation of transfection of the PGT gene, the culture medium was exchanged with the above-mentioned culture medium (neither glutamic acid nor 2-mercaptoethanol being contained therein) containing Aβ 25-35 (AnaSpec Inc.; that which was cultured at 37° C. for one week to aggregate was used) and a cultivation was carried out for 24 hours to carry out an Aβ exposure. In the meanwhile, PGE 1 (Cayman Chemical) (0.01,0.1,1 and 10 μM)was added to the culture medium together with the Aβ exposure and cultured for the same time.

5) Detection of Apoptosis

After the cells were fixed (at room temperature for 5 minutes) with a 10% neutral buffered formalin solution, they were stained with 0.01 mg/mL Hoechst 33342 (being dissolved in PBS(−); Molecular Probe) and, under a fluorescent microscope, GFAP-positive total cell numbers and apoptosis cells (the cells which were aggregated and fragmented by chromatin) were counted in random 20 fields for one example whereupon the apoptosis-positive cell percentage (%) was calculated.

2. Explanation of Figures

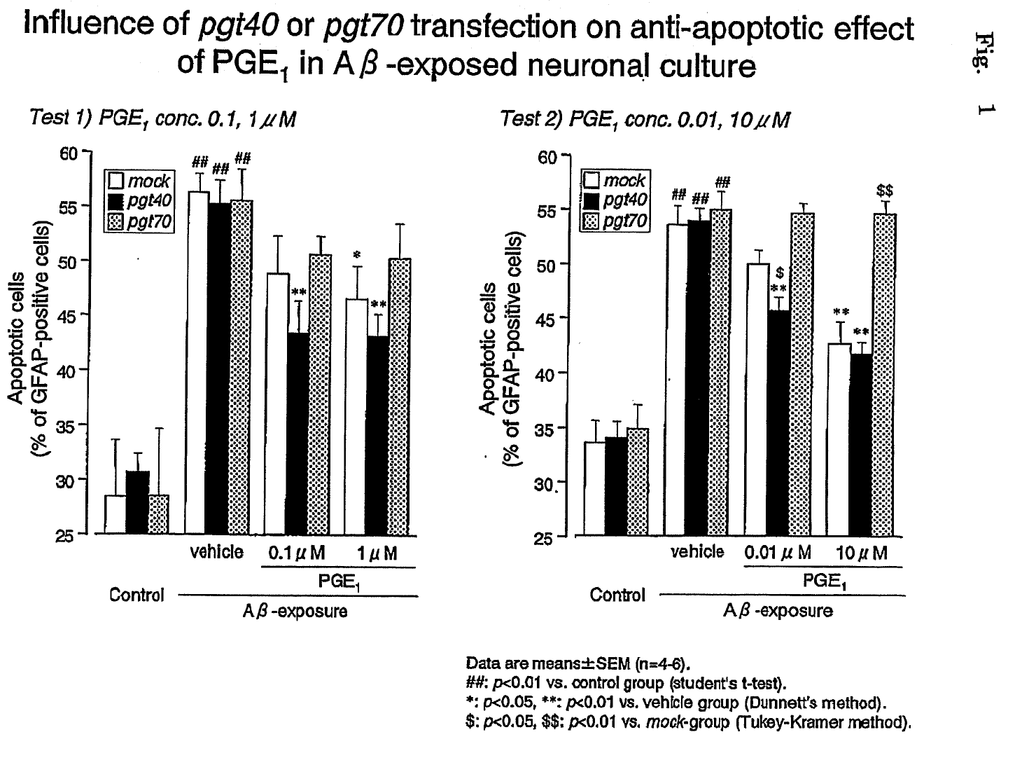

The FIG. 1 is the result shown in terms of percentage of the apoptosis-positive cells.

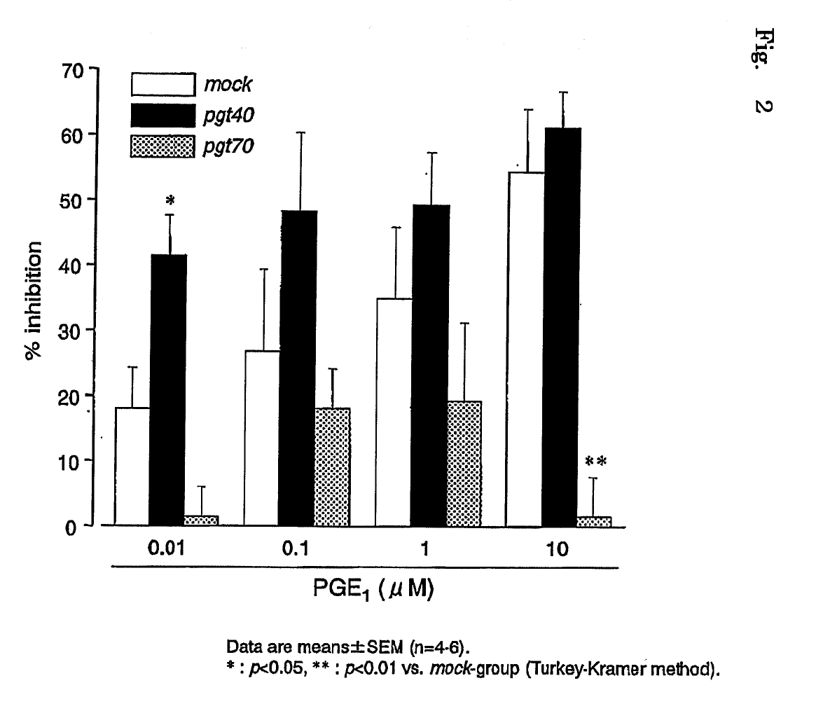

The FIG. 2 is the result shown in terms of the apoptosis inhibiting rate by taking the data in the FIG. 1 into consideration. (The values are those where the control in the FIG. 1 has an inhibiting rate of 100% and the vehicle therein has an inhibiting rate of 0%.)

The term “mock” in the tables means a blank cell. Thus, it is a cell into which no expression vector of any of PGT 40 and PGT 70 was introduced. To be more specific, in 3) of the Test Method, only the pTracer-SV 40 vector was introduced into a neuron.

3. Result

The data suggested following summaries.

1) PGT40 (human brain type) accelerate anti-apotosis action of PGE1.

2) PGT40 and PGT 70 have entirely different function for apotosis.

3) PGT40, which was found specifically in human brain, has special influences for apotosis function in brain

### INDUSTRIAL APPLICABILITY

mRNA and polynucleotide of a human brain type and polypeptide for which the said polynucleotide is coded according to the present invention are useful for clarification of action mechanism of the PGT in brain. Further, when the polynucleotide of the present invention is used, it is possible to produce the PGT of a human brain type by a genetic engineering means or, to be more specific, by a method for the transformation of appropriate host (e.g., microbes such as Escherichia coli, yeast, Bacillus subtilis, etc., animal cells and the like), a method for inducing into transgenic animals, etc. using the said polynucleotide

### Free Text of Sequence Listing

SEQ ID NO: 1 of the Sequence Listing: DNA coding for the PGT of a human lung type

SEQ ID NO: 2 of the Sequence Listing: DNA prepared using mRNA coding for the PGT of a human brain type as a template

SEQ ID NO: 3 of Sequence Listing: the PGT polypeptide of a human brain type estimated from the DNA of SEQ ID NO: 2

SEQ ID NO: 4 of Sequence Listing: a primer for the PCR derived from exon 1 of the PGT of a human lung type

SEQ ID NO: 5 of Sequence Listing: a primer for the PCR derived from exon 14 of the PGT of a human lung type

1. mRNA which codes for a prostaglandin transporter (PGT) of a human brain type having a molecular weight of 38-40 kilodantons.

2. A polynucleotide selected from the following group:
1) a polynucleotide consisting of a base sequence represented by SEQ ID NO: 2 of the Sequence Listing;
2) a complementary strand of a polynucleotide consisting of a base sequence represented by SEQ ID NO: 2 of the Sequence Listing; and
3) a polynucleotide which hybridizes to a polynucleotide consisting of a base sequence represented by SEQ ID NO: 2 of the Sequence Listing under a stringent condition.

3. A polypeptide consisting of an amino acid sequence represented by SEQ ID NO: 3 of the Sequence Listing.
